(19) United States
(12) Patent Application Publication
(10) Pub. No.: US 2007/0122481 A1
(43) Pub. Date: May 31, 2007

(54) Modified Release Compositions Comprising a Fluorocytidine Derivative for the Treatment of Cancer

(75) Inventors: Gary Liversidge, Westchester, PA (US); Scott A. Jenkins, Downingtown, PA (US)

Correspondence Address: CHRISTIAN BAUER, 3500 HORIZON DRIVE, KING OF PRUSSIA, PA, 19406, US

(73) Assignee: Elan Corporation plc, Dublin (IE)

(21) Appl. No.: 11/558,202

(22) Filed: Nov. 9, 2006

Related U.S. Application Data
(63) Continuation-in-part of application No. 11/372,857, filed on Mar. 10, 2006.
Application No. 11/372,857 is a continuation-in-part of application No. 10/827,689, filed on Apr. 19, 2004.
Application No. 10/827,689 is a continuation of application No. 10/354,483, filed on Jan. 30, 2003, now Pat. No. 6,793,936.
Application No. 10/354,483 is a continuation of application No. 10/331,754, filed on Dec. 30, 2002, now Pat. No. 6,902,742.
Application No. 10/331,754 is a continuation of application No. 09/850,425, filed on May 7, 2001, now Pat. No. 6,730,325.
Application No. 09/850,425 is a continuation of application No. 09/566,636, filed on May 8, 2000, now Pat. No. 6,228,398.
Application No. 09/566,636 is a continuation of application No. PCT/US99/25632, filed on Nov. 1, 1999.
(60) Provisional application No. 60/106,726, filed on Nov. 2, 1998.

(51) Int. Cl. A61K 31/7072; A61K 9/24

(52) U.S. Cl. 424/472; 514/49

(57) ABSTRACT

The invention relates to a multiparticulate modified release composition comprising a fluorocytidine derivative, preferably capecitabine, and a modified release component comprising a modified release coating, a modified release matrix material, or both. Following oral delivery, the composition in operation delivers the fluorocytidine derivative in a pulsatile manner at about six to about twelve hours after administration.

### FIELD OF INVENTION

The present invention relates to multiparticulate modified release compositions comprising fluorocytidine derivatives, such as capecitabine, that are suitable for use in the treatment of cancer. In particular, the present invention relates to novel dosage forms for the controlled delivery of fluorocytidine derivatives, such as capecitabine. In addition, the invention relates to a dosage package designed to enhance patient compliance and therapeutic outcomes.

### BACKGROUND OF INVENTION

It is known that many precursors of 5-fluorouracil (5-FU), also referred to as 5-FU prodrugs, are useful as anti-tumor agents. However, the bioconversion efficiency of 5-FU precursors is poor for the treatment of patients suffering from tumors due to intestinal and immunosuppressive toxicities. Modifications of such 5-FU precursors have led to the development of fluorocytidine derivatives which exhibit improved bioconversion efficiency and toxicity.

Fluorocytidine derivatives have been described in, for example, U.S. Pat. No. 4,966,891 for “Fluorocytidine Derivatives” and U.S. Pat. No. 5,472,949 for “N⁴-(Substituted-Oxycarbonyl)-5′-Deoxy-5-Fluorocytidine Compounds, Compositions and Method of Using Same” the disclosures of which are incorporated by reference herein in their entireties. U.S. Pat. No. 4,966,891 describes 5′deoxy-5-fluorocytidine derivatives, a process for their manufacture, anti-tumor compositions comprising said derivatives and its use to inhibit tumor growth in a subject. U.S. Pat. No. 5,472,949 describes N⁴-(substituted-oxycarbonyl)-5′-deoxy-5-fluorocytidine derivatives, a process for their manufacture, anti-tumor compositions comprising said derivatives and their use in the treatment of tumors in a host.

Capecitabine is a fluorocytidine derivative with the chemical name 5′deoxy-5-fluoro-N-[(pentyloxy)carbonyl]-cytidine. Capecitabine has a molecular weight of 359.35 and has the following structural formula:

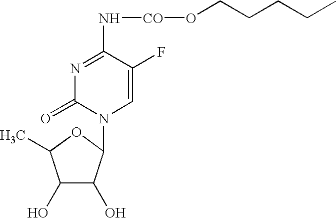

Capecitabine is a white to off-white crystalline powder with an aqueous solubility of 26 mg/ml at 20° C.

Capecitabine is enzymatically converted to 5-FU in vivo. Capecitabine is readily absorbed from the gastrointestinal tract. In the liver, a 60 kDa carboxylesterase hydrolyzes much of the compound to 5′-deoxy-5-fluorocytidine (5′-DFCR). Subsequently, cytidine deaminase, an enzyme found in most tissues including tumors, converts 5′-DFCR to 5′deoxy-5-fluorouridine (5′-DFUR). Thymidine phosphorylase then hydrolyzes 5′-DFUR to the active drug 5′-FU. Many tissues express thymidine phosphorylase. Human carcinomas, however, express the enzyme at higher concentrations than surrounding normal tissues.

Both normal and tumor cells metabolize 5-FU to 5-fluoro-2′-deoxyuridine monophosphate (FdUMP) and 5-fluoruridine triphosphate (FUTP). These metabolites cause cell injury by two different mechanisms. First, FdUMP and the folate cofactor, N⁵⁻¹⁰-methylenetetrahydrofolate, bind to thymidylate synthase (TS) to form a covalently bound ternary complex. This binding inhibits the formation of thymidylate from 2′-deoxyuridylate. Thymidylate is the necessary precursor of thymidine triphosphate, which is essential for the synthesis of DNA. Hence, a deficiency of thymidylate can inhibit cell division. Second, nuclear transcriptional enzymes can mistakenly incorporate FUTP in place of uridine triphosphate (UTP) during the synthesis of RNA. This metabolic error can interfere with RNA processing and protein synthesis, and eventually lead to cell death.

Capecitabine is regarded as a fluorocytidine derivative with high therapeutic value based on its susceptibility to hepatic carboxylesterase, oral bioavailability in monkeys and efficacy in a human cancer xenograft. Capecitabine given orally yielded substantially higher concentrations of 5-FU within tumors than in plasma or normal muscle tissue. The tumor 5-FU levels were also much higher than those achieved by intraperitoneal administration of 5-FU at equi-toxic doses. This tumor selective delivery of 5-FU ensured greater efficacy and a more favorable safety profile than with other fluoropyrimidines. In 24 human cancer xenograft models studied, capecitabine was more effective at a wider dose range and had a broader spectrum of anti-tumor activity than 5-FU, UFT or its intermediate metabolite 5′-DFUR. The susceptibility of the xenografts to capecitabine correlated with tumor dThdPase levels. Moreover, the conversion of 5′-DFUR to 5-FU by dThdPase in tumor was insufficient in a xenograft model refractory to capecitabine. In addition, the efficacy of capecitabine was enhanced by dThdPase up-regulators, such as by taxanes and cyclophosphamide and by X-ray irradiation. The efficacy of capecitabine may, therefore, be optimized by selecting the most appropriate patient population based on dThdPase status and/or by combining it with dThdPase up-regulators. Capecitabine has additional characteristics not found with 5-FU, such as potent anti-metastatic and anti-cachectic actions in mouse tumor models. Based on these profiles, capecitabine may have substantial potential in cancer treatment.

Capecitabine is offered under the registered trademark XELODA® by Hoffman-La Roche Inc. of Nutley, N.J. XELODA® is supplied as biconvex, oblong film-coated tablets for oral administration in dosages of 150 mg and 500 mg of capecitabine. The film coating contains hydroxypropyl methylcellulose, talc, titanium dioxide, and synthetic yellow and red iron oxides.

XELODA® has been proven to be effective in the treatment of colorectal cancer and breast cancer. XELODA® is indicated as a first-line treatment of patients with metastatic colorectal cancer when treatment with fluoropyrimidine therapy alone is preferred. Either XELODA® alone or XELODA® in combination with other anti-cancer agents, such as docetaxel, has proven effective in the treatment of metastatic breast cancer which is resistant to other chemotherapy regimens.

The recommended dose of XELODA® is 1250 mg/m² administered orally twice daily (morning and evening; equivalent to 2500 mg/m² total daily dose) for 2 weeks followed by a 1-week rest period given as 3-week cycles. XELODA® tablets should be swallowed with water within 30 minutes after a meal. In combination with doctaxel, the recommended dose of XELODA® is 1250 mg/m² twice daily for 2 weeks followed by a l-week rest period, combined with docetaxel at 75 mg/m² as a 1-hour intravenous infusion every 3 weeks.

Fluorocytidine derivatives are of high therapeutic value for the-treatment of cancer. Given that fluorocytidine derivatives, such as capecitabine, require oral administration twice daily, strict patient compliance is a critical factor in the efficacy of fluorocytidine derivatives in the treatment of cancer. Moreover, such frequent administration often requires the attention of health care workers and contributes to the high cost associated with treatments involving fluorocytidine derivatives, such as capecitabine. Thus, there is a need in the art for fluorocytidine derivative compositions which overcome these and other problems associated with their use in the treatment of cancer.

### SUMMARY OF THE INVENTION

It is an object of the present invention to provide a multiparticulate modified release composition containing a fluorocytidine derivative.

It is another object of the invention to provide a multiparticulate modified release composition which, in operation, delivers a fluorocytidine derivative in a unimodal manner.

It is a further object of the invention to provide a multiparticulate modified release composition containing a fluorocytidine derivative in which the active ingredient is released rapidly after an initial delay period of about six to about twelve hours.

It is yet another object of the invention to provide a multiparticulate modified release composition in an erodable, diffusion controlled, or osmotic form.

It is yet a further object of the invention to provide a multiparticulate modified release composition which, in co-administration with an immediate release form of a fluorocytidine derivative, substantially mimics the pharmacological and therapeutic effects produced by the administration of two or more immediate release dosage forms containing a fluorocytidine derivative given sequentially.

It is still another object of the invention to provide an oral dosage form comprising the multiparticulate modified release composition of the present invention.

It is still a further object of the invention to provide a method for the treatment of cancer by the administration of a therapeutically effective amount of the multiparticulate modified release composition of the present invention.

The above objects are realized by the compositions, dosage forms and methods of the present invention. According to one aspect of the invention, there is provided a multiparticulate modified release composition comprising a fluorocytidine derivative that is designed to release all of the active ingredient at about six to about twelve hours after administration. When co-administered with an immediate release dosage form comprising a fluorocytidine derivative, the resulting plasma profile is substantially similar to the plasma profile produced by the administration of two or more immediate release dosage forms given sequentially, such as the plasma profile obtained by the twice a day dosing of XELODA®.

The compositions of the present invention utilize a modified release feature to allow dosing less frequently than with conventional forms of fluorocytidine derivatives which increases patient convenience and compliance. The modified release may be achieved by the use of formulations such as, for example, erodable formulations, diffusion controlled formulations or osmotic controlled formulations. In one embodiment, the present invention relates to a multiparticulate modified release composition that, in operation, delivers a fluorocytidine derivative in a pulsatile manner. When co-administered in the evening with an immediate release dosage form comprising a fluorocytidine derivative, the composition of the present invention releases the active ingredient at about six to about twelve hours after administration so as to mimic the plasma profile obtained if that dose had been administered in the morning.

In one embodiment, the multiparticulate modified release composition comprises a fluorocytidine derivative, preferably capecitabine. The modified release may be achieved by the use of a modified-release coating, a modified release matrix material, or both. Following oral delivery, the composition in operation delivers the fluorocytidine derivative in a unimodal pulsatile manner. Preferred modified release components comprise erodable formulations, diffusion controlled formulations and osmotic controlled formulations. Preferably, the composition contains a fluorocytidine derivative in an amount from about 0.1 mg to about 1 g.

According to another aspect of the invention, there is provided an oral dosage form comprising the multiparticulate modified release composition of the present invention. The dosage forms may be provided in any suitable form such as, for example, in hard or soft gelatin capsules, or as tablets.

According to yet another aspect of the invention, there is provided a dosing package comprising one or more dosage forms of the present invention packaged together with one or more immediate release forms comprising a fluorocytidine derivative.

The present invention further provides a method for the treatment of a cancer comprising the step of administering a therapeutically effective amount of the composition of the present invention to a cancer patient in need thereof.

Advantages of the present invention include reducing the dosing frequency required by conventional multiple immediate release dosage regimes while still maintaining the benefits derived from a pulsatile plasma profile. A formulation which may be administered at reduced frequency is advantageous in terms of patient compliance. The reduction in dosage frequency made possible by utilizing the present invention would contribute to reducing health care costs by reducing the amount of time spent by health care workers on the administration of drugs.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

As used in this specification and appended claims, the singular forms “a”, “an” and “the” include plural referents unless the context clearly dictates otherwise.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixture thereof, irrespective of their size, shape or morphology.

The term “modified release” as used herein means a release which is not immediate and is taken to encompass controlled release, sustained release and delayed release.

The term “time delay” as used herein refers to the duration of time between administration of the composition and the release of the active ingredient therefrom.

As used herein, the term “active ingredient” includes one or more fluorocytidine derivatives, preferably capecitabine. The fluorocytidine derivative can be included in a microparticulate drug delivery system in which the fluorocytidine derivative is, or is entrapped within, encapsulated by, attached to, or otherwise associated with, a particulate.

The particulates of the present invention have a modified release component that may comprise a modified release coating, a modified release matrix material, or both. The modified release particulates of the present invention may be also provided in the forms disclosed in the U.S. Pat. No. 6,228,398 to Devane et al. which is incorporated by reference herein in its entirety. Following oral delivery, the composition of the present invention delivers the fluorocytidine derivative in a unimodal manner.

The modified release of the fluorocytidine derivative from the composition may be accomplished by the use of a coating, or a matrix material, or both, and results in a time delay of about six to about twelve hours between administration and the release of the fluorocytidine derivative. The duration of the time delay may be varied by altering the composition and/or the amount of the modified release coating and/or altering the composition and/or amount of modified release matrix material utilized. Suitable formulations for use in varying the time delay include erodable formulations, diffusion controlled formulations and osmotic controlled formulations. By use of such formulations, the duration of the time delay can be designed to produce a desired plasma profile.

In one embodiment, the composition can be in the form of an erodable formulation in which the structural integrity of the particulates deteriorates within the body over time. In one such embodiment, the active ingredient is released by the degradation of the modified release coating and/or the matrix materials by the action of human ingestion over a period of time.

In another embodiment, the composition can be in the form of a diffusion controlled formulation in which the particulates are dispersed in a liquid medium. One such embodiment is described in U.S. Pat. No. 6,586,006 to Roser et al. which is incorporated by reference herein in its entirety.

In another embodiment, the composition can be in the form of an osmotic controlled formulation in which the release of the active ingredient from the composition is controlled by osmosis. One such embodiment is described in U.S. Pat. No. 6,110,498 to Rudnic et al. which is incorporated by reference herein in its entirety and which discloses a the release of a therapeutic agent having limited water solubility in solubilized form. The delivery system comprises a core that is free of swellable polymers and comprises nonswelling solubilizing agents and wicking agents. The solubilized therapeutic agent is delivered through a passageway in the semipermeable coating of the tablet.

U.S. Pat. No. 5,814,979 B2 also to Rudnic et al. which is incorporated by reference herein in its entirety describes an osmotic pharmaceutical delivery system comprising: (a) a semi-permeable wall that maintains its integrity during pharmaceutical delivery and which has at least one passage therethrough; (b) a single, homogeneous composition within said wall, which composition consists essentially of: (i) a pharmaceutically active agent; (ii) at least one non-swelling solubilizing agent which enhances the solubility of the pharmaceutically active agent; (iii) at least one non-swelling osmotic agent; and (iv) a non-swelling wicking agent dispersed throughout the composition which enhances the surface area contact of the pharmaceutical agent with the incoming aqueous fluid.

The composition may further comprise additional components such as, for example, an enhancer compound or a sensitizer compound in order to modify the bioavailability or the therapeutic effect of the fluorocytidine derivative.

As used herein, the term “enhancer” refers to a compound which is capable of enhancing the absorption and/or bioavailability of an active ingredient by promoting net transport across the gastrointestinal tract in an animal, such as a human. Suitable enhancers include, either alone or in combination, medium chain fatty acids as well as salts, esters, ethers and other derivatives thereof including glycerides and triglycerides; ionic and non-ionic surfactants such as those that can be prepared by reacting ethylene oxide with a fatty acid, a fatty alcohol, an alkylphenol or a sorbitan or glycerol fatty acid ester; cytochrome P450 inhibitors; bile salts and acids: micelles chelators; P-glycoprotein inhibitors and the like.

The plasma profile associated with the administration of the composition may be described as “pulsatile profile” in which one or more peaks of plasma concentration of an active ingredient are observed. A pulsatile profile containing one peak may be described as “unimodal,” and a pulsatile profile containing two peaks separated by a lower concentration trough may be described as “bimodal,” and a pulsatile profile containing more than two peaks in which each adjacent pair is separated by a lower concentration trough may be described as “multimodal.”

Conventional frequent dosage regimes in which an immediate release (IR) dosage form is administered at periodic intervals typically give rise to a pulsatile plasma profile. A peak in the plasma drug concentration is observed after administration of each IR dose with troughs developing between consecutive administration time points. The plasma profile produced by concurrent administration of the modified release composition along with an immediate release composition is substantially similar to the plasma profile produced by the administration of two or more immediate release dosage forms given sequentially.

In one embodiment, the active fluorocytidine derivative is capecitabine and the composition, in operation, delivers the capecitabine in a unimodal manner. In another embodiment, the active fluorocytidine derivative is capecitabine and the modified release composition is co-administered in the evening with an immediate release form of capecitabine and produces a plasma profile which substantially mimics the plasma profile obtained by the sequential administration of two immediate release doses as, for instance, in a typical anti-cancer drug treatment regimen.

The release characteristics of the composition may be varied by, for example, modifying the amount and/or type of coating and/or matrix material used in the composition. As noted above, the release profiles may be further controlled by the selective use of erodable formulations, diffusion controlled formulations or osmotic controlled formulations. Similarly, the plasma concentration curve produced by the administration of the composition may be varied by, for example, modifying the amount and/or type of coating and/or matrix material used in the composition. Depending on the duration of the time delay of the modified release composition, the pulses in the plasma profile produced by the concurrent administration of modified release and immediate release forms may be well-separated and clearly defined (e.g., when the time delay of the modified release composition is long) or the pulses may be superimposed to a degree (e.g., when the time delay of the modified release composition is short).

In one embodiment, the modified release composition according to the present invention has a single modified release component in which the modified release is realized by the use of a modified release coating, a modified release matrix material, or both. In operation, co-administration of such a modified release composition with an immediate release form results in a pulsatile plasma profile characterized by a first peak in the plasma profile associated with the immediate release form and a second peak in the plasma profile associated with the modified release component. Embodiments of the invention in which more than one modified release component is present exhibit additional peaks in the plasma profile.

The administration of an immediate release form and at least one modified release form is advantageous when it is desirable to deliver two (or more) pulses of active ingredient without the need for sequential administration of two (or more) dosage units. Additionally, in the case of cancer treatment, it is particularly useful to produce such a bimodal or multimodal plasma profile. For example, a typical treatment regime using capecitabine consists of administration of two doses of an immediate release dosage formulation given about 6 to about 12 hours apart. This type of regime has been found to be therapeutically effective and is widely used.

Any coating material which modifies the release of the fluorocytidine derivative in the desired manner may be used. In particular, coating materials suitable for use in the practice of the invention include polymer coating materials, such as cellulose acetate phthalate, cellulose acetate trimaletate, hydroxypropylmethylcellulose phthalate, poly(vinyl acetate) phthalate, ammonio methacrylate copolymers such as those sold under the Trade Mark Eudragit® RS and RL, poly(acrylic acid) and polyacrylate and methacrylate copolymers such as those sold under the Trade Mark Eudragit® S and L, poly(vinyl acetaldiethylamino) acetate, hydroxypropylmethylcellulose acetate succinate, shellac, hydrogels and gel-forming materials such as carboxyvinyl polymers, sodium alginate, sodium carmellose, calcium carmellose, sodium carboxymethyl starch, poly(vinyl alcohol), hydroxyethylcellulose, methylcellulose, gelatin, starch, and cellulose based cross-linked polymers in which the degree of crosslinking is low so as to facilitate adsorption of water and expansion of the polymer matrix, hydoxypropylcellulose, hydroxypropylmethylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethylcellulose, swellable hydrophilic polymers, poly(hydroxyalkyl methacrylate) (m. wt. ˜5 k-5,000 k), polyvinylpyrrolidone (m. wt. ˜10 k-360 k), a swellable mixture of agar and carboxymethylcellulose, copolymers of maleic anhydride and styrene, ethylene, propylene or isobutylene, polysaccharides such as acacia, karaya, tragacanth, algins and guar, polyacrylamides, Polyox® polyethylene oxides (m. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®, Edward Mandell C. Ltd.), hydrophilic polymers such as polysaccharides, methyl cellulose, sodium or calcium carboxymethylcellulose, nitrocellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. The term “modified release matrix material” as used herein includes hydrophilic polymers, hydrophobic polymers and mixtures thereof which are capable of modifying the release of the fluorocytidine derivative dispersed therein. Modified release matrix materials suitable for the practice of the present invention include but are not limited to microcrystalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

The modified release composition according to the present invention may be incorporated into any suitable dosage form which facilitates release of the active ingredient in a pulsatile manner. In one embodiment, the dosage form comprises a fluorocytidine derivative-containing particulates which are filled into suitable capsules, such as hard or soft gelatin capsules. Alternatively, the fluorocytidine derivative-containing particulates may be compressed (optionally with additional excipients) into mini-tablets which may be subsequently filled into capsules. Another suitable dosage form is that of a tablet. The fluorocytidine derivative-containing particulates making up the composition of the invention may further be included in rapidly dissolving dosage forms such as an effervescent dosage form or a fast-melt dosage form.

The amount of fluorocytidine derivative present in the dosage form is a therapeutically effective amount which will vary according to, among other things, the specific type of cancer being treated, the sensitivity of the patient, as well as other factors known to the skilled artisan. In one embodiment, the amount of fluorocytidine derivative present in the dosage form is from about 0.1 mg to about 1 g, preferably from about 150 mg to about 500 mg.

The present invention further provides a method for the treatment of cancer comprising the step of administering a therapeutically effective amount of the composition of the present invention. In one such embodiment, the composition comprises capecitabine that is delivered in a pulsatile manner.

### EXAMPLE 1

### Multiparticulate Modified Release Composition Containing Capecitabine

A multiparticulate modified release composition according to the present invention is prepared as follows.

(a) Preparation of Capecitabine-Containing Particles.

A solution of capecitabine (50:50 racemic mixture) is prepared according to any of the formulations given in Table 1. The capecitabine solution is then coated onto nonpareil seeds to a level of approximately 16.9% solids weight gain using, for example, a Glatt GPCG3 (Glatt, Protech Ltd., Leicester, UK) fluid bed coating apparatus to form the capecitabine-containing particles.

TABLE 1

Immediate release intermediate
Amount, % (w/w)

| Ingredient | (i) | (ii) |
|---|---|---|
| Capecitabine | 13.0 | 13.0 |
| Polyethylene Glycol 6000 | 0.5 | 0.5 |
| Polyvinylpyrrolidone | 3.5 | |
| Purified Water | 83.5 | 86.5 |

(b) Preparation of Modified Release Capecitabine-Containing Particles

Capecitabine containing modified release particles are prepared by coating the capecitabine-containing particles prepared according to Example 1(a) above with a modified release coating solution as detailed in Table 2. The capecitabine-containing particles are coated to varying levels up to approximately 30% weight gain using, for example, a fluid bed apparatus.

TABLE 2
Modified release component coating solutions
Amount, % (w/w)

| Ingredient | (i) | (ii) | (iii) | (iv) | (v) | (vi) | (vii) | (viii) |
|---|---|---|---|---|---|---|---|---|
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Eudragit ® | — | — | — | — | — | — | 25.0 | — |
| L 12.5 | | | | | | | | |
| Polyvinyl- | — | — | — | 0.35 | 0.3 | — | +113 | — |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl- | — | — | — | +113 | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.44 | — | — | — |
| Talc¹ | — | 16.0 | 5.9 | — | 16.3 | — | 2.8 | 2.25 |

¹Talc is simultaneously applied during coating for formulations in column (i), (iv) and (vi).

(c) Encapsulation of Modified Release Particles

The modified release particles prepared according to Example 1(a) and (b) above are encapsulated in size 2 hard gelatin capsules to an overall 150 mg dosage strength using a Bosch GKF 4000S encapsulation apparatus.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

We claim:

1. A multiparticulate modified release composition comprising a fluorocytidine derivative and a modified release coating or, alternatively or additionally, a modified release matrix material, such that the composition following oral delivery to a subject delivers the fluorocytidine derivative in a pulsatile manner.

2. The composition of claim 1, wherein the fluorocytidine derivative is capecitabine.

3. The composition of claim 1, wherein the modified release component comprises an erodable formulation.

4. The composition of claim 1, wherein the modified release component comprises a diffusion controlled formulation.

5. The composition of claim 1, wherein the modified release component comprises an osmotic controlled formulation.

6. The composition of claim 1, wherein the modified release component comprises a modified release coating.

7. The composition of claim 1, wherein the modified release component comprises a modified release matrix material.

8. The composition according to claim 1, wherein the amount of said fluorocytidine derivative is from about 0.1 mg to about 1 g.

9. The composition according to claim 8, wherein the amount of said fluorocytidine derivative is about 150 mg or about 500 mg.

10. The composition according to claim 1, wherein substantially all of the fluorocytidine derivative is released at about six to about twelve hours after administration to a patient.

11. The composition according to claim 1, wherein the modified release component comprises a pH-dependent polymer coating capable of releasing a pulse of the fluorocytidine derivative following a time delay of about six to about twelve hours after administration to a patient.

12. The composition according to claim 11, wherein the polymer coating comprises methacrylate copolymers.

13. The composition according to claim 12, wherein the polymer coating comprises a mixture of methacrylate and ammonio methacrylate copolymers.

14. The composition according to claim 13, wherein the ratio of methacrylate to ammonio methacrylate copolymers is approximately 1:1.

15. A dosage form comprising the composition of claim 1.

16. A dosage form comprising the composition of claim 2.

17. The dosage form of claim 15, wherein the composition is provided in a hard or soft gelatin capsule.

18. The dosage form of claim 17, wherein the composition is in the form of mini-tablets.

19. The dosage form of claim 15, wherein the composition is compressed to form a tablet.

20. The dosage form of claim 15, wherein the composition is provided in a rapidly dissolving dosage form.

21. The dosage form according to claim 20, wherein the composition is provided as a fast-melt tablet.

22. A method for the treatment of cancer comprising the step of administering a therapeutically effective amount of the composition according to claim 1.

23. A method for the treatment of cancer comprising the step of administering a therapeutically effective amount of the composition according to claim 2.

24. The method according to claim 23, wherein substantially all of the capecitabine is released about 6 to about 12 hours after administration of the composition.

25. The method according to claim 24 further comprising the step of co-administering a therapeutically effective amount of an immediate release composition of capecitabine.

26. The method according to claim 25, wherein the compositions are administered once-a-day.

27. The method according to claim 25, wherein the compositions are administered in the evening.

28. A combination dosage package comprising an immediate release dosage form of capecitabine and the dosage form according to claim 16, wherein the dosage forms are packaged together.
